(19) United States
(12) Patent Application Publication
(10) Pub. No.: US 2012/0301520 A1
(43) Pub. Date: Nov. 29, 2012

(54) DIRECTLY COMPRESSIBLE MAGNESIUM HYDROXIDE CARBONATE

(75) Inventors: Marianne Dohmen, Gachenbach (DE); Ralf Fielder, Beerfelden (DE); Heike Mueller, Darmstadt (DE); Jens Klatyk, Griesheim (DE); Hans Kurt Peth, Alsbach-Haehnlein (DE); Guenter Moddelmog, Reinheim (DE); Thorsten Wedel, Stockstadt/Rhein (DE)

(73) Assignee: MERCK PATENT GESELLSCHAFT MIT BESCHRANKTER HAFTUNG, Darmstadt (DE)

(21) Appl. No.: 13/576,674

(22) PCT Filed: Jan. 7, 2011
(86) PCT No.: PCT/EP2011/000045

§ 371 Date: Aug. 2, 2012

(30) Foreign Application Priority Data
Feb. 3, 2010 (EP) 10001099.0

(51) Int. Cl. C01F 11/18; C01F 5/24; A61K 33/10; B29C 43/02; A61K 9/14; A61P 3/02

(52) U.S. Cl. 424/400; 423/430; 424/686; 264/319; 428/402

(57) ABSTRACT

The present invention relates to a directly compressible magnesium hydroxide carbonate and to a process for the preparation thereof, and to the use thereof.

The present invention relates to a directly compressible magnesium hydroxide carbonate and to a process for the preparation thereof, and to the use thereof.

### PRIOR ART

Basic magnesium carbonate or magnesium hydroxide carbonate, having the chemical composition: 4MgCO3×Mg(OH)2×5H2O can be prepared from magnesium carbonate (MgCO₃). Magnesium carbonate is a white powder which has very low solubility in water. It is formed from aqueous solution only if the latter contains a large amount of excess carbonic acid. Magnesium carbonate can crystallise with 5, 3 and 1 mol of water of crystallisation and is gradually decomposed to basic magnesium carbonate on boiling with water. Corresponding processes for the preparation have been known for some time.

For the preparation of pharmaceutical compositions and for industrial applications, basic magnesium carbonate (magnesia alba) is much more frequently used than pure magnesium carbonate. Magnesium hydroxide carbonate is usually obtained by precipitation from a magnesium sulfate solution using soda.

Magnesium hydroxide carbonate is a snow-white, very light, loose, water-insoluble powder which dissolves in acids much more quickly than magnesite. It is therefore generally used as starting material for the preparation of other magnesium compounds.

Furthermore, magnesium hydroxide carbonate is used in antacids against gastric hyperacidity, as tooth powder, as wound powder, as antidote against poisoning by acids, arsenic and metal salts, for the preparation of powders, cleaning powders, etc. However, it is also used as filler for paints, paper, rubber, as refractory material, for heat insulation and the like.

In particular, magnesium hydroxide carbonate is frequently used as mineral substance for the magnesium enrichment of foods, pharmaceuticals and dietetic preparations. It is preferably also employed as constituent in (compressed) tablets, such as, for example, in chewable and effervescent tablets, since it has a relatively high magnesium content and is available at a favourable price. In effervescent tablets, it additionally serves as carbon dioxide source for producing the effervescent effect. Compressed tablets of this type are preferably produced by direct tableting processes, i.e. without prior granulation steps.

However, pulverulent magnesium hydroxide carbonate known to date cannot be tableted directly without special additives or special pretreatment owing to its poor flow properties and owing to the lack of compressibility.

EP 0 460 923 describes the preparation of a basic magnesium carbonate for which BET surface areas of 10-70m²/g are claimed. The examples disclosed have BET surface areas of up to max. 38 m²/g (Table 1). For these materials, an average particle size of 1 to 50 μm is claimed, while the average particle size confirmed by examples is only in the range 3.8 to 6.0 μm (Table 1). However, the only corresponding products that are actually commercially available are from Lehmann & Voss “Phar Magnesia MC Type A granulate”, which have a BET surface area of 32 m²/g.

In EP 0 460 923, the basic magnesium carbonate is prepared by a reaction from magnesium sulfate heptahydrate with sodium carbonate with addition of a “crystallising assistant” (Table 1). In order to achieve a high BET surface area and a high pore volume, it is absolutely necessary here to introduce the reactants into the reaction vessel as solids, since otherwise a product according to the invention cannot be obtained. The use of reaction products which are already pre-dissolved is explicitly excluded.

If the compression properties of the pure magnesium hydroxide carbonates which are available on the market and are claimed to be “directly compressible” are investigated, these are not convincing. Better tableting properties are achieved by the combination of the brittle magnesium salt with a plasticising component, for example in the form of starch. Although such compositions can be compressed better, they have a reduced magnesium content owing to the addition of binder. In addition, they can no longer be characterised by a simple pharmacopoeia monograph. In particular, the latter can result in registration or documentation problems for the users in the preparation of pharmaceutical formulations. In addition, the preparation of these compositions is quite complex, due to additional process steps, such as, for example, a requisite granulation.

Object

The object of the present invention is therefore to provide an inexpensive magnesium hydroxide carbonate which is simple to prepare, which has the highest possible magnesium content and meets the purity requirements of conventional pharmacopoeias and which can be employed directly and can be compressed in compression processes without further additives. A further object of the present invention is to provide an inexpensive process which is simple to carry out for the preparation of a directly compressible magnesium hydroxide carbonate of this type.

Achievement of the Object

The object of the present invention is achieved by precipitating magnesium hydroxide carbonate from aqueous solutions of the starting salts in a continuous process, and subsequently subjecting it to a drying process. The modified preparation process gives a magnesium hydroxide carbonate having a modified appearance, which has particularly advantageous pharmaceutical formulation properties and exhibits an unusually high BET surface area compared with commercially available products. This novel material according to the invention can be directly tableted very well and, in its purity criteria, meets the requirements of Ph Eur, BP, USP and E 504 for so-called heavy magnesium hydroxide carbonate (heavy, basic magnesium carbonate).

In particular, the present object is achieved by the provision of directly compressible magnesium hydroxide carbonate, (heavy, in accordance with the purity requirements of Ph Eur, BP, USP and E 504), which is directly compressible by compression with a pressing force of in the range from 10 kN to 20 kN to give tablets having hardnesses in the range >80 N to >200 N and a friability <0.2% by weight. This magnesium hydroxide carbonate can likewise be compressed by compression with a pressing force of in the range from 10 kN to 20 kN to give tablets having a friability <0.1% by weight. The present invention thus relates, in particular, to a magnesium hydroxide carbonate of this type which has a BET surface area of at least 44 to 70 m²/g, preferably greater than 50 m²/g. At the same time, it has a bulk density in the range 0.25-0.80 g/ml, in particular in the range from 0.40 to 0.60 g/ml, and a tapped density in the range 0.35-0.90 g/ml, in particular in the range from 0.50 to 0.80 g/ml. For the terms bulk density and tapped density, the terms bulk weight and tapped weight are also customary. Furthermore, it preferably has average particle diameters (laser; D_{0.50}) in the range between 20 and 60 μm.

In accordance with the invention, this magnesium hydroxide carbonate can be prepared in a continuous reaction, where a warmed solution of a magnesium salt in which the magnesium content in the solution is 2-11% by weight, particularly preferably 3-6% by weight, and a warmed solution of an alkali-metal or alkaline-earth metal carbonate in which the carbonate content in the solution is 2-18% by weight, preferably 3-15% by weight, are mixed in a tubular reactor at a temperature in the range from 60 to 70° C., and the pH is kept in the range 8.5-9.0. The precipitated magnesium hydroxide carbonate is subsequently filtered off and then dried in a convection dryer to a content of 40 to 43.5% by weight (calculated as MgO).

The present invention also relates to the use of the directly compressible magnesium hydroxide carbonate in accordance with one or more of claims 1 to 6 as constituent of tablet formulations in accordance with claims 10 to 12.

In particular, the present invention also relates to a process for the preparation of the directly compressible magnesium hydroxide carbonate according to the invention. This process is characterised in that

a) a warmed solution of a magnesium salt in which the magnesium content is 2-11% by weight, preferably 3-10% by weight, in particular 3-6% by weight, and a warmed solution of an alkali-metal or alkaline-earth metal carbonate in which the carbonate content is 2 -18% by weight, preferably 3-16% by weight, particularly preferably 3-15% by weight and particularly preferably 7-10% by weight, are pumped continuously into a tubular reactor and mixed so that a temperature of 60 to 70° C. and a pH in the range 8.5 -9.0 becomes established in the reaction solution, and the magnesium hydroxide carbonate formed precipitates out and

b) the precipitated product is filtered off from the product-containing reaction mixture, if necessary after being allowed to settle for some time, and dried. The subsequent drying is preferably carried out in a convection dryer, preferably a fluidised-bed dryer. The filtered-off product is dried here to a content of 40 to 43.5% by weight (calculated as MgO). Before the product is separated off, it is advantageous to allow the product-containing reaction mixture to settle at a temperature in the range 55-65° C. for a time of 10-45 minutes.

### DETAILED DESCRIPTION OF THE INVENTION

The magnesium hydroxide carbonate according to the invention gives the formulation pharmacist a product which is optimised with respect to the direct-tableting properties, enabling (compressed) tablets having a highly dosed magnesium content to be produced inexpensively in a simple manner. In particular, this magnesium hydroxide carbonate can be employed directly for tableting without pretreatment in a particular granulation step.

For the preparation of the product according to the invention, an alkali-metal or alkaline-earth metal carbonate and a soluble magnesium salt, for example a chloride, sulfate or similar soluble salt in the form of warmed solutions, which are pumped separately into a suitable reactor, are per se brought to reaction in a continuous process. The product formed precipitates out and, optionally after additional treatment steps, is converted into a pulverulent form via a drying process, for example by convection drying, preferably by drying in the fluidised bed. In addition, no further treatment is necessary. The material obtained in this way meets the purity criteria of the foods and pharmaceuticals industry and is distinguished by high BET surface areas in the range 44-70 m²/g. The direct tableting can be carried out simply by mixing with a tableting assistant which is conventional in the pharmaceutical industry, and subsequent compression. The use of the magnesium hydroxide carbonate prepared in this way enables higher tablet hardnesses to be obtained at the same pressing forces than in the case of the use of directly compressible magnesium hydroxide carbonates commercially available to date.

The products according to the invention are preferably prepared in a continuous process. In order to carry out this process, the starting materials are dissolved in water. The starting materials are carbonates, in particular alkali-metal or alkaline-earth metal carbonates, and suitable magnesium salts, such as chloride, sulfate or similar soluble salts, particularly preferably magnesium chloride.

An aqueous solution in which the respective carbonate component is present in a concentration of about 2-18% by weight, preferably about 3-15% by weight, particularly preferably 7-10% by weight, is prepared from the carbonates with warming. An aqueous solution having a magnesium content in the range 2-11% by weight, preferably 3-10% by weight, particularly preferably 3-6% by weight, is prepared from the magnesium salt, preferably magnesium chloride. The contents of the solutions are in practice adjusted via a correlation with the density of the solutions, where the density can be determined by various methods known to the person skilled in the art. For industrial use, electro-acoustic methods, for example by means of a densimeter with vibration transducer, are to be preferred, since they can be carried out simply and can be evaluated directly online. The preparation of the starting solutions can in this way be automated in a simple manner in combination with suitable dispensing devices. Depending on the magnesium salt employed, it is dissolved in water in an exothermic reaction. If it is necessary, the magnesium salt solution obtained is warmed. In order to guarantee a fast reaction, the pH in the magnesium salt solution is set in a range between 4.5 and 6.0, preferably in a range between 5-5.5. If necessary, the pH can be adjusted by addition of the complementary acid or a suitable base, such as MgO.

The solutions obtained are subsequently mixed continuously with one another under controlled conditions. To this end, the solutions are warmed and mixed with one another while maintaining the temperature in a virtually constant range. At the same time, the pH is set and controlled in a certain range. Without addition of a “crystallising assistant”, a pure product is obtained in this way which meets the requirements of the pharmacopoeias, and is directly compressible.

In accordance with the invention, this precipitation reaction can be carried out in any reaction vessel which is suitable for carrying out continuous reactions in the liquid phase and in which reliable mixing of the supplied media can take place. However, the use of tubular reactors, into which the pre-warmed starting solutions are pumped continuously and from which the product-containing reaction mixture formed flows out continuously, depending on the pump speed, after an average residence time which can be set subsequently, has proven particularly suitable for the preparation of the magnesium hydroxide carbonate according to the invention. Average residence times in the reactor of 4 to 20 min, in particular 7-15 min, have proven particularly suitable in the experiments carried out.

Precipitated, crystalline product which is already formed by reaction of the salts is present in the reaction mixture obtained in this way and can be separated off per se directly by known methods and dried.

For carrying out the continuous reaction, the use of a tubular reactor having an internal diameter of 300 mm and a length of 3300 mm has proven particularly successful. However, the dimensions of a suitable tubular reactor of this type can be modified in accordance with the desired throughputs so long as suitable mixing of the reaction liquids is maintained. Corresponding modifications are readily possible for the person skilled in the art who is familiar with the scale-up or scale-down of chemical reaction apparatus through suitable experiments.

In order to carry out the reaction in the tubular reactor which is preferred here, the separately warmed salt solutions are introduced into the tubular reactor by means of pumps in such a way that flow takes place through the tubular reactor with an amount of liquid of about 1300 l/h, preferably about 1500 l/h.

The desired magnesium hydroxide carbonate is precipitated while maintaining a pH in the reaction solution in the range from 8.5 to 9.0 and a temperature in the range 60-70° C. The pH automatically becomes established per se at the desired value on mixing of the starting solutions. If necessary, the pH can be adjusted by addition of small amounts of magnesium oxide or a corresponding acid. This can also take place during the reaction if the reactor is fitted with corresponding measurement and metering devices. However, it is more favourable for the pH of the starting solutions to be set in advance in such a way that the pH becomes established in the desired range automatically on mixing of the starting solutions.

Series of experiments have shown that improved product properties can be achieved if the product-containing reaction mixture obtained is collected in suitable storage vessels and allowed to settle for some time. In this way, both the reaction of the salts and also the precipitation of the desired product can be completed. Precipitated, crystalline product can be separated off from the reaction mixture directly or after a corresponding settling time. It has proven advantageous per se for the suspensions obtained by the reaction to be collected and allowed to settle. This also has the advantage that the suspension obtained can be fed uniformly to the filtration unit.

The separation-off of product can be carried out in a manner known to the person skilled in the art, for example by centrifugation or filtration. Separation-off by filtration is particularly suitable. Belt filtration has proven suitable for continuous separation-off of product.

The liquid to be filtered is fed continuously to a filter sheet which consists of a suitable nonwoven or woven fabric. The particles to be separated off are retained on the filter sheet, and the liquid freed from particles is discharged to disposal. The residue remaining on the filter sheet forms a so-called filter cake, which can be washed with pure water, but also with suitable solvents, for purification while still on the filter belt. Immediately after the filtration, the washed filter cake is fed to drying. The entire operation can be carried out continuously and fully automatically without the liquid stream having to be interrupted.

The drying step following the separation-off of the precipitated magnesium hydroxide carbonate can be carried out in various ways known to the person skilled in the art. In order to prevent baking of the product obtained, the immediate drying is preferably carried out by convection drying. The drying is particularly preferably carried out in a fluidised bed. For this purpose, the moist magnesium hydroxide carbonate separated off is transferred into the fluidised bed of the corresponding dryer and dried. The air blown into the dryer can have a temperature of <250° C. here. However, the drying is preferably carried out at moderate temperatures. The moist magnesium hydroxide carbonate is dried in this way to a content of 40 to 43.5% by weight (calculated as MgO).

The drying is preferably carried out using a suitable convection dryer or fluidised-bed dryer operated with warmed air which preferably has a temperature in the range from 70 to 140° C. For example, a fluidised-bed dryer of the WST/WSG type from Glatt (Germany) is suitable for this purpose. However, comparable commercially available equipment can also be employed.

For drying, a certain amount of filtered-off product are initially introduced in a fluidised-bed apparatus [GPCG 5/Glatt (Germany)]. Warm air is passed through the material until the latter breaks down into its fine components. If the feed air is supplied here with a temperature in a range from about 70° to <250° C., preferably in the range from 70 to 140° C., the temperature of the exhaust air becomes established in a correspondingly lower range for a certain amount of air flowing through. The amount of air flowing through is preferably adjusted to about 370 to 450 Nm³/h. The exhaust-air temperature becomes established to about 35 to 65° C. if the temperature of the feed air is in the preferred range. The material usually begins to fluidise after drying for about 15 to 20 minutes. At this time, the amount of exhaust air can be reduced to 135 to 165 Nm³/h with maintenance of the temperature profile.

The drying is continued until the product has the desired humidity. Any obstinate lumps present in the product can be eliminated by sieving through a suitable sieve. A sieve having a mesh width of 710 μm or finer can be employed for this purpose. The drying process is terminated, and a content determination in accordance with Ph. Eur. is carried out. If the content determination (calculated as MgO) shows excessively low values, drying must be continued.

The magnesium hydroxide carbonate having improved properties with respect to the tableting properties which is characterised in accordance with Ph. Eur., USP, BP, E 504 as “basic heavy magnesium carbonate” is obtained in the manner described.

Even without the addition of a binder or without prior additional granulation, the product obtained has very good tableting properties, i.e. the magnesium hydroxide carbonate, which is brittle per se, can, even at low pressing forces, be converted into tablets having good hardnesses, which in turn have significantly lower abrasion than conventional comparable tablets. The magnesium hydroxide carbonate according to the invention thus behaves significantly better than comparable commercially available DC magnesium carbonates.

Even compared with the commercially available grades, which are magnesium carbonates which have been converted into a directly compressible form by addition of a binder (10% of starch), the tableting properties of the material according to the invention are at least equivalent or better.

In addition, experiments have shown that the material according to the invention, besides a significantly greater BET surface area, also has a significantly increased BET pore volume than commercially available products. These modified properties are externally associated with a modified particle size and particle structure and, in the use for the production of tablets, with improved compressibility. The magnesium hydroxide carbonate obtained, which meets the requirements of Ph Eur, BP, USP and E 504, can be compressed directly to give tablets in a simple manner without further tableting assistants and, on compression with a pressing force of 10 kN to 20 kN, results in tablets having hardnesses in the range from >80 N to >200N at the same time as a friability of <0.2% by weight, in particular <0.1% by weight.

In detail, this improved material has enlarged BET surface areas in the range from 44 to 70 m²/g, preferably greater than 50 m²/g. However, the BET pore volume of this product is also significantly increased compared with commercially available DC magnesium carbonate. The magnesium hydroxide carbonate characterised in this way has a bulk density in the range 0.25-0.80 g/ml, in particular in the range from 0.40 to 0.60 g/ml, and a tapped density in the range 0.35-0.90 g/ml, in particular in the range from 0.50 to 0.80 g/ml.

By contrast, however, the particle structure is significantly coarser than in the case of corresponding commercially available products: the D_{(0.50)} values of the measured average particle sizes are in the range from 20 to 60 μm, in particular in the range from 24 to 60 μm.

The directly compressible magnesium hydroxide carbonate prepared in accordance with the invention can, owing to its improved properties, be used as constituent in active compound-containing tablet formulations, chewable tablets and lozenges, effervescent tablets, effervescent powders, in capsule formulations or in powder preparations for magnesium enrichment. However, it is also highly suitable for the preparation of tablet formulations which comprise vitamins, mineral substances, trace elements, functional food constituents or for the preparation of tablet formulations comprising active compounds, or of tablet formulations which comprise synthetic or natural dyes, natural and/or nature-identical aromas and/or other flavouring substances, such as, for example, from the group aspartame, saccharin, acesulfame K, neohesperidine, sucralose, thaumatin and stevioside, or fruit aromas, fruit acids, flavouring plant extracts and pharmaceutical or dietetic active compounds.

Methods

The instruments and methods used for the characterisation of the material properties are shown below:
1. The bulk density is determined in accordance with DIN EN ISO 60: 1999 (German version). The data in the present description, the examples and the tables are in “g/ml”
2. The tapped density of the products obtained is determined in accordance with DIN EN ISO 787-11: 1995 (German version). The data in the present description, the examples and the tables are in “g/ml”
3. The angle of repose of the products obtained is determined in accordance with DIN ISO 4324 (German version). The data in the present description, the examples and the tables are in “degrees”
4. The tableting testing is carried out as follows:
   492.5 g of the material to be tested for its tableting properties are mixed with 7.5 g of Parteck LUB MST (vegetable magnesium stearate) EMPROVE exp Ph Eur, BP, JP, NF, FCC Art. No. 1.00663 (Merck KGaA, Germany); the magnesium stearate is deposited in advance via a 250 μm sieve, and mixed for 5 minutes in a sealed stainless-steel container (capacity: about 2 l, height: about 19.5 cm, diameter: about 12 cm; external dimensions) in a laboratory tumble mixer (Turbula, Willy A. Bachofen, Switzerland). The compression to give 500 mg tablets (11 mm punch, round, flat, with bevel) is carried out on a Korsch EK 0-DMS instrumented eccentric tableting machine (Korsch, Germany) with the Catman 5.0 evaluation system, Hottinger Baldwin Messtechnik—HBM (Germany).
   Depending on the pressing force tested (nominal settings: 5+/−1, 10+/−2, 20+/−2 and 30+/−2 kN; the effectively measured actual values are indicated in the examples), at least 100 tablets are produced for evaluation of the pressing data and their pharmaceutical formulation characteristic numbers.
5. Determination of the tablet hardness, diameter and heights: Erweka TBH 30 MD; Erweka (Germany); average data (arithmetic means) from in each case 20 tablet measurements per pressing force
6. Determination of the tablet abrasion: friability tester Erweka (Germany); instrument parameters and performance of the measurements in accordance with Ph. Eur. 6th Edition “Friability of uncoated tablets”
7. Tablet weight: average value (arithmetic means) from the weighing of 20 tablets per pressing force; balance: Mettler AT 201, Mettler (Germany)
8. Determination of the drying loss in accordance with Ph. Eur. 6th Edition, main work 2008, Volume 1, page 70, method 2.2.32 d) at 130° C.
9. Determination of the particle-size distribution:
   Laser scattering with wet dispersal Mastersizer 2000 Ver. 5.22, Serial Number: 34403-97 with Hydro 2000S (A) dispersion unit from Malvern Instruments Ltd. (UK); dispersion medium: demineralised water, RI 1.330; pump speed: 2000 rpm; stirrer speed: 2000 rpm; ultrasonic duration: 1 sec, ultrasonic level: 100%, tray type: general purpose; back-ground time: 7500 msec, measurement time: 7500 msec; obscuration limits: 10.0-20.0%; evaluation. Fraunhofer; performance in accordance with ISO 13320-1 and the information in the technical manual and the instrument manufacturer's specifications
10. Determination of the BET surface area and BET pore volume (single point adsorption total pore volume):
   Performance and evaluation in accordance with the literature “BET Surface Area by Nitrogen Adsorption”, S. Brunauer et al. (Journal of American Chemical Society, 60, 9, 1938), the instrument manufacturer's specifications and DIN ISO 9277; instrument: ASAP 2420 V 1.03a Z from Micromeritics Instrument Corporation (USA); nitrogen; volumetric method; sample weight about 3 g +/−10%, with sample preparation (drying by heating at 3.0° C./min. to the target temperature 50° C.: 5 hours/50° C.)
11. Content determination of the carbonate- and magnesium-containing batches via a density determination by means of a densimeter with vibration transducer: Anton Paar DMA 4500 densimeter (Austria); measurement at 70° C.;
   Performance in accordance with determination: “Relative density, Ph. Eur. 6th Edition, main work 2008, Volume 1, page 33, method 2.2.5 and European standard EN ISO 15212-1: 1999 German version: densimeters in accordance with the oscillator principle
12. Content determination of magnesium (calculated as MgO): in accordance with Ph. Eur. 6th Edition “Heavy, basic magnesium carbonate”

The references to generally valid purity specifications for “heavy” grade magnesium hydroxide carbonate made in the preceding description relate to the reviews, monographs and directives mentioned below:
1. Ph Eur: “Heavy, basic magnesium carbonate” or “Magnesii subcarbonas ponderosus” European pharmacopoeia 6th Edition, main work 2008, Volume 3, monographs K-Z, official German edition, page 3143-3144 Deutscher Apotheker Verlag Stuttgart, Govi-Verlag-Pharmazeutischer Verlag GmbH Eschborn ISBN 978-3-7692-3962-1
2. BP: “Heavy magnesium carbonate”, British Pharmacopoeia (BP) 2009, Volume II, General Notices Monographs, Medicinal and Pharmaceutical Substances (J-Z), ISBN 978 0 11 322799 0, page 1263-1264
3. USP: “Magnesium carbonate”, The United States Pharmacopeia 2009, USP 32-NF 27, Volume 3, USP Monographs M-Z, ISBN 1-889788-69-2, USP 32, page 2828-2829
4. E 504: “E 504 (ii) Magnesium hydroxide carbonate”, Commission Directive 2008/84/EC of 27. Aug. 2008 for stipulating specific purity criteria for food additives other than dyes and sweeteners, Official Journal of the European Union dated 20.09.2008 DE, page L 253/119

### LIST OF TABLES AND FIGURES

Table 1: Reaction conditions, content (of Examples A-E according to the invention)

Tables 2 and 3: DC magnesium hydroxide carbonate, heavy: pharmaceutical formulation properties, bulk density, tapped density, flow angle, particle-size distribution, BET surface area, pore volume (Examples A-E according to the invention compared with powder F and compared with commercial DC magnesium carbonates without binders G, H, and with binders I-L)

Table 4: DC magnesium hydroxide carbonate, heavy: tableting data (Examples A-E according to the invention compared with powder F and compared with commercial DC magnesium carbonates without binders G,H)

Table 5: DC magnesium hydroxide carbonate, heavy: tableting data (Examples A-E according to the invention compared with powder F and compared with commercial DC magnesium carbonates comprising 10% of starch I-L)

FIG. 1: DC magnesium hydroxide carbonate, heavy: tableting data (Examples A-E according to the invention vs. powder F vs. commercial DC magnesium carbonates without binders G,H), tablet hardnesses as a function of pressing force, data from Table 4

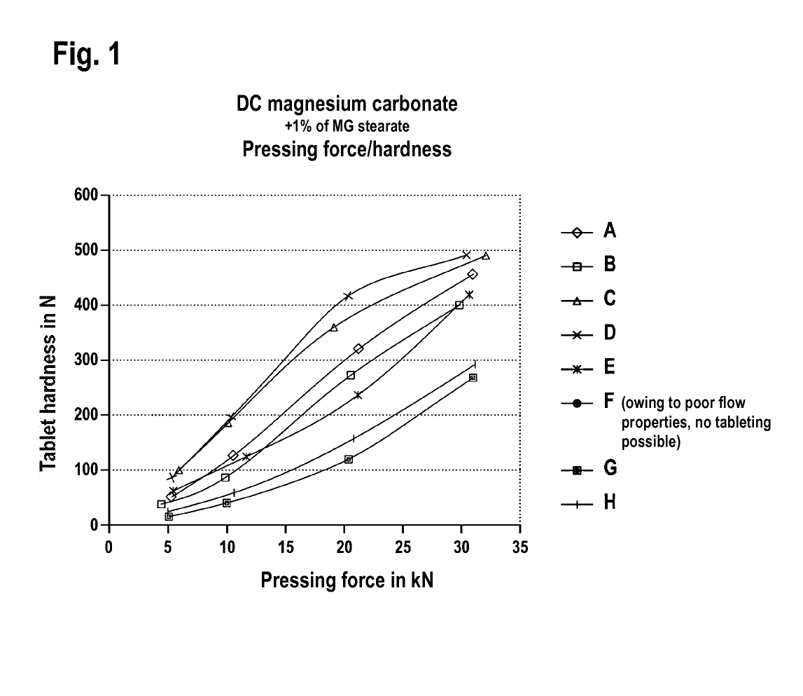

FIG. 2: DC magnesium hydroxide carbonate, heavy: tableting data (Examples A-E according to the invention compared with powder F and compared with commercial DC magnesium carbonates comprising 10% of starch I-L), tablet hardnesses as a function of pressing force; data from Table 5

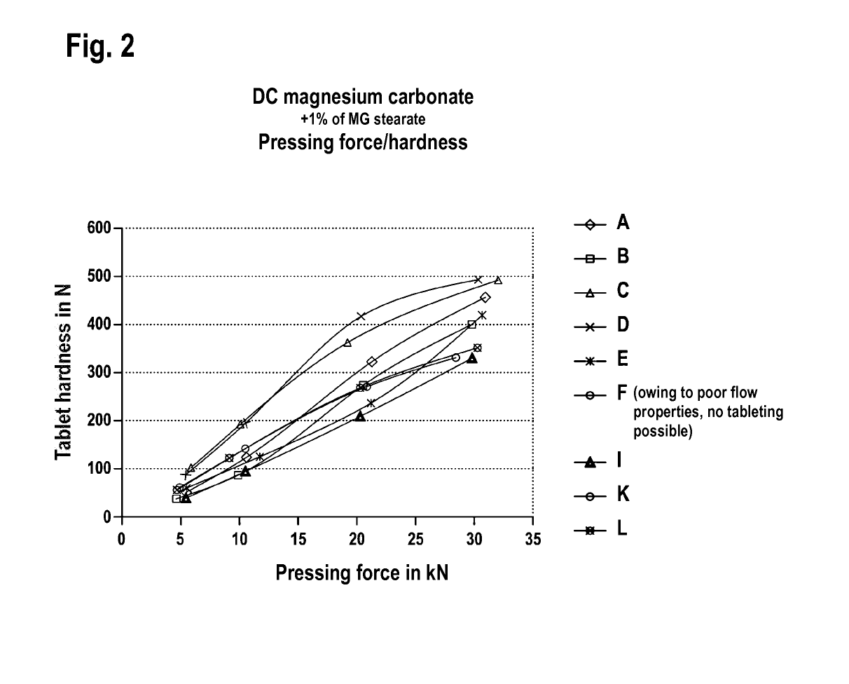

In the further description, examples of the process according to the invention for the preparation of the magnesium hydroxide carbonate according to the invention which are within the scope of protection of the present invention are given for better understanding and in order to explain the invention. These examples also serve to illustrate possible process variants. Owing to the general validity of the inventive principle described, however, the examples are not suitable for reducing the scope of protection of the present application to these alone.

The temperatures given in the examples and description and in the claims are always in ° C. Unless indicated otherwise, content data are given as % by weight or weight ratios.

Furthermore, it goes without saying to the person skilled in the art that, both in the examples given and also in the remainder of the description, the component amounts present in the compositions always only add up to 100% by weight, mol % or % by volume, based on the composition as a whole, and cannot exceed this, even if higher values could arise from the per cent ranges indicated in tables. Unless indicated otherwise, % data are % by weight, with the exception of ratios, which are shown in volume data.

### EXAMPLES

Preparation of Magnesium Hydroxide Carbonate

The preparation of magnesium hydroxide carbonate in the grade described is achieved by continuous precipitation of the product from sodium carbonate solution and magnesium chloride solution, separation-off of the reaction products and drying:

A solution is prepared from sodium carbonate, Na₂CO₃, with warming. The preparation of the magnesium chloride solution proceeds as a strongly exothermic reaction on use of magnesium chloride anhydrate, MgCl₂. The pH is adjusted to pH 5-5.5 by addition of a small amount of 37% hydrochloric acid or magnesium oxide.

For the continuous preparation, a hot 3-15% carbonate solution and a hot 2-10% Mg solution are introduced into a tubular reactor by means of pumps. (The solutions are prepared in such a way that the stated % by weight of carbonate, or magnesium ions are in each case present in the solutions, based on the total weight of the solutions.) The components are brought to reaction in the molar ratio 0.6-0.8 mol of magnesium ions to 1 mol of carbonate ions. The content adjustments of the solutions are in practice carried out via a correlation with the density of the solutions. The reactor used has the dimension of an internal diameter of 300 mm to a length of 3300 mm. An average residence time in the reactor of 7 to 15 min was determined for the reaction.

The precipitation is carried out immediately with observance of the pH (pH 8.5-9.0) and the temperature of 60-70° C. without additional heat source. The suspension formed is temporarily stored in a container for uniform product feed to the filtration unit and for completion of the reaction, before it is separated off on a belt filter unit and washed in accordance with the specified chemical quality parameters.

The filter cake forming there is dried using hot air at <250° C., preferably at 70-140° C., in order to obtain the product corresponding to the composition 4MgCO₃*Mg(OH)₂*4H₂O.

In this way, the magnesium hydroxide carbonate which is characterised in accordance with Ph. Eur., USP, BP, E 504 as “basic heavy magnesium carbonate” is obtained.

Data of various experimental runs, in each of which the salt content was adjusted in a controlled manner, are reproduced in Table 1 below for comparison.

TABLE 1
Reaction conditions, contents,
Examples A-E according to the invention

| | Example A | Example B | Example C | Example D | Example E |
|---|---|---|---|---|---|
| Carbonate content | 15.3 | 3.0 | 7.3 | 9.6 | 6.9 |
| [%] | | | | | |
| Magnesium content | 2.4 | 3.8 | 3.6 | 4.1 | 9.7 |
| [%] | | | | | |
| Temperature of carbonate | 82 | 80 | 80 | 80 | 80 |
| solution [° C.] | | | | | |
| Temperature of magnesium | 70 | 71 | 70 | 70 | 70 |
| salt solution | | | | | |
| [° C.] | | | | | |
| pH of magnesium salt | 5.0 | 4.7 | 5.2 | 5.2 | 4.9 |
| solution | | | | | |
| Precipitation temperature | 68 | 65 | 67 | 61 | 66 |
| [° C.] | | | | | |
| Precipitation pH | 9 | 9 | 9 | 9 | 9 |
| Drying loss after filtration | 48 | 53 | 50 | 57 | 54 |
| [%] | | | | | |
| Drying temperature | 130 | 130 | 70 | 90 | 130 |
| (feed air) [° C.] | | | | | |

Description of the Drying for Example C:

2000 g of the moist material taken from the belt filter (50-60% of adhering water fractions) are initially introduced in a GPCG 5/Glatt (Germany) fluidised-bed apparatus. Warm air (feed-air temperature 70° to 70.6° C.; exhaust-air temperature 38.6° to 42.7° C.; at an air amount of 396 to 416 Nm³/h)) is passed through the material until the latter breaks down into its fine components—any obstinate lumps are eliminated by sieving through a 710 μm sieve. If the material begins to fluidise after drying for about 20 minutes, the amount of exhaust air is reduced to 140 to 160 Nm³/h (the feed-air and exhaust-air temperatures remain unchanged) and dried under these conditions for a further 30 minutes. After a total drying time of 50-55 minutes (and a rel. humidity of 20-21% in the exhaust air at 36° C.), the process is terminated, and a content determination in accordance with Ph. Eur. is carried out. Practical yield 780 g (small material losses in the apparatus, for example in the filters and adhering to the apparatus walls). Should the content determination (calculated as MgO) indicate a lower content, drying must be continued.

Description of the Drying for Example D:

2000 g of the moist material taken from the belt filter (50-60% of adhering water components) are initially introduced in a GPCG 5 Glatt (Germany) fluidised-bed apparatus. Warm air (feed-air temperature 87.3° to 90.3° C.; exhaust-air temperature 39.5° to 48.2° C.; at an air amount of 399 to 427 Nm³/h)) is passed through the material until the latter breaks down into its fine components—any obstinate lumps are eliminated by sieving through a 710 μm sieve. If the material begins to fluidise after drying for about 20 minutes, the amount of exhaust air is reduced to 142 to 159 Nm³/h (the feed-air and exhaust-air temperatures remain unchanged) and dried under these conditions for a further 30 minutes. After a total drying time of 50-55 minutes (and a rel. humidity of 15-16% in the exhaust air at 40° C.), the process is terminated, and a content determination in accordance with Ph. Eur. is carried out. Practical yield 780g (small material losses in the apparatus, for example in the filters and adhering to the apparatus walls). Should the content determination (calculated as MgO) indicate a lower content, drying must be continued.

TABLE 2

| | A | B | C | D | E | F |
|---|---|---|---|---|---|---|
| Bulk density [g/ml] | 0.43 | 0.55 | 0.42 | 0.42 | 0.53 | 0.49 |
| Tapped density [g/ml] | 0.57 | 0.73 | 0.59 | 0.58 | 0.75 | 0.70 |
| Flow angle [°] | 42.1 | 41.0 | 40.2 | 39.8 | 45.0 | 47.4 |
| Particle-size distribution | | | | | | |
| [% by vol.] | | | | | | |
| D (0.10) | 10.8 | 8.7 | 9.7 | 9.8 | 6.4 | 3.8 |
| D (0.25) | 34.4 | 21.2 | 19.1 | 19.6 | 15.0 | 15.8 |
| D (0.50) | 59.6 | 34.1 | 31.5 | 32.4 | 24.6 | 36.1 |
| D (0.75) | 88.4 | 49.7 | 48.0 | 49.2 | 36.8 | 60.5 |
| D (0.90) | 118.1 | 66.0 | 66.5 | 67.7 | 50.3 | 85.4 |
| BET surface area [m²/g] | 50 | 60 | 67 | 70 | 44 | 16 |
| BET pore volume [cm³/g] | 0.28 | 0.29 | 0.28 | 0.30 | 0.20 | 0.08 |

TABLE 3

| | G | H | I | K | L |
|---|---|---|---|---|---|
| Bulk density | 0.63 | 0.59 | 0.45 | 0.39 | 0.53 |
| [g/ml] | | | | | |
| Tapped density | 0.77 | 0.74 | 0.55 | 0.47 | 0.68 |
| [g/ml] | | | | | |
| Flow angle | 33.8 | 35.1 | 30.3 | 28.4 | 39.9 |
| [°] | | | | | |
| Particle-size | | | | | |
| distribution [% | | | | | |
| by vol.] | | | | | |
| D (0.10) | 2.1 | 1.8 | 14.3 | 5.8 | 6.8 |
| D (0.25) | 6.0 | 3.3 | 29.3 | 17.1 | 19.6 |
| D (0.50) | 16.9 | 9.6 | 45.0 | 37.2 | 39.2 |
| D (0.75) | 34.3 | 46.0 | 63.3 | 59.9 | 61.5 |
| D (0.90) | 65.0 | 99.1 | 81.8 | 82.8 | 84.0 |
| BET surface | 12 | 32 | 15 | 6 | 6 |
| area | | | | | |
| [m²/g] | | | | | |
| BET pore volume | 0.09 | 0.21 | 0.09 | 0.03 | 0.04 |
| [cm³/g] | | | | | |

TABLE 4

| | Pressing force | | | |
|---|---|---|---|---|
| | [kN] | | Tablet hardness | Friability |
| Sample | Nominal | Actual | n. 1 day [N] | [%] |
| A | 5 | 5.4 | 52.0 | 0.92 |
| | 10 | 10.6 | 124.6 | 0.14 |
| | 20 | 21.3 | 321.5 | 0.06 |
| | 30 | 31.0 | 457.0 | 0.01 |
| B | 5 | 4.6 | 37.6 | 1.11 |
| | 10 | 9.9 | 88.3 | 0.05 |
| | 20 | 20.6 | 272.9 | 0 |
| | 30 | 29.8 | 397.1 | 0 |
| C | 5 | 5.9 | 103.8 | 0.01 |
| | 10 | 10.1 | 189.3 | 0.01 |
| | 20 | 19.2 | 361.2 | 0 |
| | 30 | 32.1 | 491.6 | 0 |
| D | 5 | 5.4 | 88.1 | 0.11 |
| | 10 | 10.5 | 194.7 | 0 |
| | 20 | 20.4 | 415.9 | 0 |
| | 30 | 30.4 | 492.2 | 0 |
| E | 5 | 5.5 | 61.0 | 0.66 |
| | 10 | 11.7 | 124.5 | 0.07 |
| | 20 | 21.2 | 238.0 | 0 |
| | 30 | 30.7 | 419.1 | 0 |
| F | 5 | Owing | poor flow properties | no tableting |
| | 10 | to | | possible! |
| | 20 | | | |
| | 30 | | | |
| G | 5 | 5.2 | 15.1 | 6.92 |
| | 10 | 10.1 | 39.9 | 1.20 |
| | 20 | 20.4 | 120.2 | 0.22 |
| | 30 | 31.0 | 269.9 | 0.11 |
| H | 5 | 5.0 | 24.2 | 3.70 |
| | 10 | 10.5 | 59.1 | 0.34 |
| | 20 | 20.8 | 158.6 | 0.08 |
| | 30 | 31.0 | 290.5 | 0.04 |

TABLE 5

| | Pressing force [kN] | | Tablet hardness | Friability |
|---|---|---|---|---|
| Sample | Nominal | Actual | n. 1 day [N] | [%] |
| A | 5 | 5.4 | 52.0 | 0.92 |
| | 10 | 10.6 | 124.6 | 0.14 |
| | 20 | 21.3 | 321.5 | 0.06 |
| | 30 | 31.0 | 457.0 | 0.01 |
| B | 5 | 4.6 | 37.6 | 1.11 |
| | 10 | 9.9 | 88.3 | 0.05 |
| | 20 | 20.6 | 272.9 | 0 |
| | 30 | 29.8 | 397.1 | 0 |
| C | 5 | 5.9 | 103.8 | 0.01 |
| | 10 | 10.1 | 189.3 | 0.01 |
| | 20 | 19.2 | 361.2 | 0 |
| | 30 | 32.1 | 491.6 | 0 |
| D | 5 | 5.4 | 88.1 | 0.11 |
| | 10 | 10.5 | 194.7 | 0 |
| | 20 | 20.4 | 415.9 | 0 |
| | 30 | 30.4 | 492.2 | 0 |
| E | 5 | 5.5 | 61.0 | 0.66 |
| | 10 | 11.7 | 124.5 | 0.07 |
| | 20 | 21.2 | 238.0 | 0 |
| | 30 | 30.7 | 419.1 | 0 |
| F | 5 | Owing | poor flow properties | no tableting |
| | 10 | to | | possible! |
| | 20 | | | |
| | 30 | | | |
| I | 5 | 5.4 | 39.4 | 0.47 |
| | 10 | 10.5 | 92.0 | 0.07 |
| | 20 | 20.3 | 207.4 | 0.01 |
| | 30 | 29.8 | 329.5 | 0.08 |
| K | 5 | 4.8 | 58.9 | 0.16 |
| | 10 | 10.4 | 139.5 | 0.09 |
| | 20 | 20.7 | 268.3 | 0.06 |
| | 30 | 28.3 | 329.3 | 0.08 |
| L | 5 | 4.7 | 56.5 | 0.18 |
| | 10 | 9.2 | 121.5 | 0.11 |
| | 20 | 20.4 | 267.5 | 0.08 |
| | 30 | 30.4 | 350.8 | 0.05 |

Commercially Available Grades of Powder Products and of Directly Compressible Magnesium Hydroxide Carbonates, Heavy, Employed for Comparative Experiments:
F: Magnesium hydroxide carbonate, heavy, extra pure, Ph EUR, BP, USP, E 504, Merck KGaA, Darmstadt [Germany], Art. No. 1.05829, batch K38796529 (this is a powder product without claimed DC properties)
G: NutriMag MC DC magnesium carbonate heavy, pharmaceutical grade, granulated, in the purity in accordance with BP, USP, Ph. Eur, CALMAGS GmbH, Lüneburg [Germany], batch: 308075060
H: Pharmagnesia MC type A granules, magnesium carbonate, heavy, granules, pharmaceutical, EP, E504, Lehmann & Voss, Hamburg [Germany], Art. No. 2420230, batch: 0805-089
I: SCORAMAG DC 90ST comprising 10% of starch, Scora, Caffiers [France], batch: 07/348/C414
K: Magnesium carbonate DC 90S/C, granules, comprising about 10% of corn starch, Dr. Paul Lohmann, Emmerthal [Germany], Art. No. 501003036270, batch: 234298
L: Magnesium carbonate DC 90S/F, granules, comprising about 10% of corn starch, Dr. Paul Lohmann, Emmerthal [Germany], Art. No. 501003036280, batch: 138252

1.-12. (canceled)

13. Directly compressible magnesium hydroxide carbonate, (heavy in accordance with the requirements of Ph Eur, BP, USP and E 504), characterised in that it has a BET surface area of at least 44 to 70 m²/g, a bulk density of 0.40 to 0.60 g/ml and a tapped density of 0.50 to 0.80 g/ml.

14. Directly compressible magnesium hydroxide carbonate according to claim 13, characterised in that it has an average particle diameter (laser; D_{0.50}) in the range between 20 and 60 μm, in particular in the range from 24 to 60 μm.

15. Directly compressible magnesium hydroxide carbonate according to claim 13, characterised in that it has a BET surface area of greater than 50 m²/g.

16. Directly compressible magnesium hydroxide carbonate according to claim 13, obtainable by continuous reaction in a tubular reactor, in which magnesium carbonate is precipitated from a warmed solution obtained by mixing a solution of a magnesium salt having a magnesium content in the solution of 2-11% by weight and a solution of an alkali-metal or alkaline-earth metal carbonate having a carbonate content in the solution of 2-18% by weight at a temperature in the range from 60 to 70° C. at a pH of 8.5-9.0 and filtered off and subsequently subjected to drying to a content of magnesium hydroxide carbonate of 40 to 43.5% by weight, calculated as MgO, in a convection dryer.

17. Directly compressible magnesium hydroxide carbonate according to claim 16, obtainable by continuous reaction in a tubular reactor, where precipitation is carried out from a warmed solution obtained by mixing a solution having a magnesium content in the solution of 3-6% by weight and a solution having a carbonate content in the solution of 2-18% by weight.

18. Tablets which have hardnesses in the range >80 N to >200 N and a friability <0.2% by weight and which have been produced using a magnesium hydroxide carbonate of claim 13 by compression with a pressing force in the range from 10 kN to 20 kN.

19. Tablets according to claim 18, having a friability <0.1% by weight.

20. Process for the preparation of a directly compressible magnesium hydroxide carbonate according to claim 13, characterised in that
a) a warmed solution of a magnesium salt and a warmed solution of an alkali-metal or alkaline-earth metal carbonate are pumped continuously into a tubular reactor and mixed with one another, where the solution of the magnesium salt has a magnesium content of 2-11% by weight and the solution of the alkali-metal or alkaline-earth metal carbonate has a carbonate content of 2-18% by weight, so that a temperature of 60 to 70° C. and a pH in the range 8.5-9.0 become established in the reaction solution, and the magnesium hydroxide carbonate formed is precipitated out and
b) the product precipitated out of the product-containing reaction mixture, if necessary after being allowed to settle for some time, is filtered off and dried.

21. Process according to claim 20, characterised in that a warmed solution of a magnesium salt having a magnesium content of 3-6% by weight and a solution of an alkali-metal or alkaline-earth metal carbonate having a carbonate content of 3-15% by weight are pumped continuously into a tubular reactor and mixed.

22. Process according to claim 20, characterised in that the filtered-off product is dried in a fluidised-bed dryer or in a convection dryer.

23. Process according to claim 20, characterised in that the filtered-off product is dried to a content of 40 to 43.5% by weight of magnesium carbonate (calculated as MgO).

24. A process for the production of tablets having a hardness >80 N and a friability <0.2% by weight by pressing a directly compressible magnesium hydroxide carbonate according to claim 13 with a pressing force of 10 kN to 20 kN.

25. A process for the production of tablets having a hardness >80 N and a friability <0.1% by weight by pressing a directly compressible magnesium hydroxide carbonate according to claim 13 with a pressing a force of 10 kN to 20 kN.

26. A composition comprising a directly compressible magnesium hydroxide carbonate according to claim 13 as constituent in active compound-containing tablet formulations, chewable tablets and lozenges, effervescent tablets, effervescent powders, in capsule formulations or in powder preparations for magnesium enrichment.

27. A composition comprising a directly compressible magnesium hydroxide carbonate according to claim 13 in a tablet formulations comprising vitamins, mineral substances, trace elements, functional food constituents or active compounds.

28. A composition comprising a directly compressible magnesium hydroxide carbonate according to claim 13 in a tablet formulations comprising synthetic or natural dyes, natural and/or nature-identical aromas and/or other flavouring substances, such as, for example, from the group aspartame, saccharin, acesulfame K, neohesperidine, sucralose, thaumatin and stevioside, or fruit aromas, fruit acids, flavouring plant extracts and pharmaceutical or dietetic active compounds.
